(19) United States
(12) Patent Application Publication
(10) Pub. No.: US 2024/0374824 A1
(43) Pub. Date: Nov. 14, 2024

(54) MEDICAL DELIVERY DEVICE

(71) Applicant: HOFFMANN-LA ROCHE INC., Little Falls, NJ (US)

(72) Inventors: Paul Graham HAYTON, Bristol (GB); Jonathan Paul RIDLEY, Bristol (GB); Mark Digby TEUCHER, Bristol (GB); Christian FISCHLEIN, Struer (DK)

(21) Appl. No.: 18/292,242

(22) PCT Filed: Sep. 29, 2022
(86) PCT No.: PCT/EP2022/077139

§ 371 Date: Jan. 25, 2024

(30) Foreign Application Priority Data
Sep. 29, 2021 (EP) 21199690.5

(51) Int. Cl. A61M 5/178; A61M 5/31; A61M 5/315

(52) U.S. Cl. CPC A61M 5/1782; A61M 5/3129; A61M 5/31501; A61M 5/31511; A61M 5/31528; A61M 5/31536; A61M 5/31553; A61M 2005/31508; A61M 2005/3151; A61M 2205/582

(57) ABSTRACT

A medical delivery device includes a barrel assembly with a hollow interior and a first thread arrangement, a rod assembly with a second thread arrangement and a plunger rod element extending into the hollow interior of the barrel assembly, and a dosage chamber formed in the hollow interior of the barrel assembly with a variable volume limited by the plunger rod element. The barrel assembly and the rod assembly are rotatable relative to each other. The first thread arrangement of the rod assembly and the second thread arrangement of the barrel assembly engage such that rotation of the barrel assembly and the rod assembly relative to each other moves the rod assembly along a longitudinal axis of its plunger rod assembly causing the volume of the dosage chamber to vary. The device further includes a rotation inhibiting formation coupled to the barrel assembly and the rod assembly.

### TECHNICAL FIELD

The present invention relates to a medical delivery device according to the preamble of independent claim 1. Such medical delivery devices having a barrel assembly with a hollow interior and a first thread arrangement, a rod assembly with a second thread arrangement and a plunger rod element having a longitudinal axis and extending into the hollow interior of the barrel assembly, and a dosage chamber formed in the interior of the barrel assembly with a variable volume limited by the plunger rod of the rod assembly, wherein the barrel assembly and the rod assembly are rotatable relative to each other, and wherein the first thread arrangement of the rod assembly and the second thread arrangement of the barrel assembly engage such that rotation of the barrel assembly and the rod assembly relative to each other moves the rod assembly along the longitudinal axis of its plunger rod assembly causing the volume of the dosage chamber to vary, can be used for dosing an individual amount of a drug substance and then administering the individually dosed amount to a patient, e.g. by injection. In particular, such medical delivery devices allow to be operated by patients themselves without requiring expertise from a physician.

### BACKGROUND ART

Delivering a liquid or other fluid out of a container is required in many medical applications and performed in a plurality of different ways. Particularly where it is essential that the liquid is comparably precisely provided, specific devices are commonly used. For example, liquid pharmaceutical substances are often provided in glass or plastic vials which are closed by a septum or rubber plug and a cap clamped around it or another similar seal cover. Conventionally, for delivering the pharmaceutical substance out of vials, syringes are used. Thereby, a needle of the syringe penetrates the septum or cover and the pharmaceutical substance is withdrawn into the syringe through its needle. Once transferred into the syringe, the pharmaceutical substance is delivered in an appropriate manner. For example, the pharmaceutical substance can be, e.g., subcutaneously or intramuscularly, injected or it can be orally applied or provided as droplets, e.g., in the eyes or nose of the patient.

Delivering liquids from vials or containers by means of syringes usually is comparably difficult. It typically makes it necessary that an educated person such as a doctor or a nurse is involved. In particular, in cases where the dosage of liquid delivered has to be comparably precise such as when comparable small volumes as in a range of ten microliter to about one milliliter are involved patients are typically not reliably capable of performing the delivery themselves when using a syringe or a similar device, i.e., self-administration can be challenging for the user. However, self-administration of liquids or medicaments is beneficial in many therapeutic applications since the effort for the patient and the costs of the therapy can be extensively reduced.

For improving this situation, there are devices used which allow for more conveniently delivering a comparably precise volume of liquids. For example, it is known to provide medicaments in prefilled syringes which can be administered by the patients themselves. However, such prefilled syringes are often not preferred for plural reasons. For example, producing prefilled syringes is comparably complicated and expensive compared to vials in terms of manufacturing. Or, syringes have to be provided with plural possible dosages suitable for different applications and patients which makes manufacture additionally cumbersome. Other examples of delivery devices are injection pens which are often used in therapy of diabetes.

An alternative delivery device is described in U.S. Pat. No. 6,607,508 B2, i.e., an automatic medicament delivery device having a cylindrical syringe barrel into which a plunger rod extends from one side. The other side of the syringe barrel is equipped with a thread onto which a needle assembly can be screwed. The plunger rod has a vial seat into which a vial can be retained. The plunger rod is further equipped with a pathway longitudinally extending throughout the entire plunger rod. Radially from the plunger rod pins extend which inter-engage with slots of a dose barrel surrounding the section of the plunger rod having the pins. By turning the dose barrel via a dose ring the plunger rod translates and a volume is created between the plunger rod and the thread side of the syringe barrel. Induced by this movement a medicament is transferred from the vial through the pathway into the volume. The turning of the dose barrel into an opposite direction is blocked by a ratchet mechanism which ensures that no liquid can be pressed back through the pathway. The device further has a spring driven and automatic needle injecting arrangement for delivering the medicament from the volume through a needle screwed to the thread of the syringe barrel. During medicament delivery the plunger rod is automatically turned in the opposite direction by a spring force and the volume is reduced. Thereby, the medicament is pressed through the needle.

Another medical delivery device improving simplicity and efficiency of use, particularly for precisely dosing and for administering a liquid drug substance after dosing, is shown in WO 2017/102742 A1. This medical delivery device comprises a rod element with a hollow body portion coaxially extending and surrounding a stem. The body portion has arm sections each of which at one end being fixed to the rest of the body portion. Each of the arm sections is equipped with a pin projecting towards the stem in an essentially radial direction. At an axial front end of the stem a rubber stopper is mounted. Further, the device comprises a dosage member with a hollow chamber cylinder. The outer surface of the chamber cylinder is equipped with a thread, wherein the pins of the body portion are engaging the thread. The interior of the chamber cylinder receives the stem and the rubber stopper of the rod element. At its front end the chamber cylinder passes over into a male coupling structure and a delivery needle projecting from the male coupling structure. Furthermore, the medical delivery device comprises a dial unit forming a vial seat in its interior. The seat is equipped with a spike. The dial unit is releasably connected to the dosage member. In use of the device, a vial is arranged in the vial seat such that the spike pierces a cover of the vial and accesses its interior. The spike is in fluid communication with the interior of the chamber cylinder via the delivery needle. For dosing a liquid from the vial into the medical delivery device, the dial unit together with the chamber cylinder are rotated such that the pins of the rod element travel along the thread of the chamber cylinder. Thereby, the stem and the rubber stopper move backwardly and a dosage chamber is generated inside the chamber cylinder. More specifically, the backward movement of the rubber stopper establishes a negative pressure in the dosage chamber such that liquid is withdrawn from the vial via the spike and the needle into the dosage chamber.

Even though such known medical delivery devices are improving the situation with respect to a convenient handling, they may not allow dosing on a comparably high level of precision. In particular, in cases where it is necessary to dose comparably precise small amounts of liquid drug substances or to provide dosages with a comparably high accuracy, known medical deliver device are typically not appropriate. The lack of dosing precision is caused often by the fact that at the end of dosing, i.e., when stopping rotation, the negative pressure inside the dosing chamber slightly pulls the stem into the dosage chamber. This may result in forwarding a small amount of liquid out of the dosage chamber or in slight deviation of the indicated dosing amount.

Therefore, there is a need for a medical deliver device allowing an improved accurate dosing.

### DISCLOSURE OF THE INVENTION

According to the invention this need is settled by a medical delivery device as it is defined by the features of independent claim 1. Preferred embodiments are subject of the dependent claims.

In particular, the invention is a medical delivery device comprising a barrel assembly, a rod assembly and a dosage chamber. The barrel assembly has a hollow interior and a first thread arrangement. The rod assembly includes a second thread arrangement and a plunger rod element having a longitudinal axis and extending into the hollow interior of the barrel assembly. The dosage chamber is formed in the interior of the barrel assembly with a variable volume limited by the plunger rod element of the rod assembly.

The term “assembly” as used in connection with the invention relates to a single piece element or a multi-component structure. It may establish a unit having at least in an application step a uniform function or purpose. The hollow interior can be embodied in any suitable component of the barrel assembly.

The barrel assembly and the rod assembly are rotatable relative to each other, particularly about the longitudinal axis of the plunger rod element. In connection with various components of the medical delivery device, the term “rotatable relative to each other” relates to any rotational movement. For example, the barrel and rod assemblies can be rotate relative to each other either by rotating the barrel assembly and the rod assembly standing still, by rotating the rod assembly and the barrel assembly standing still, or by rotating both the barrel assembly as well as the rod assembly.

The first thread arrangement of the barrel assembly and the second thread arrangement of the rod assembly engage such that rotation of the barrel assembly and the rod assembly relative to each other moves the rod assembly along the longitudinal axis of its plunger rod element causing the volume of the dosage chamber to vary. More specifically, by the rotation, the first and second thread arrangements may travel along each other which may induce a linear relative movement of the barrel and rod assemblies. Thus, by means of the thread arrangements, a rotational movement can be transformed in a well-defined and precise linear movement along the longitudinal axis. Such linear movement can be appropriate for accurately varying the volume of the dosage chamber and thereby accurately dosing a liquid such as a liquid drug substance into the dosage chamber.

The term “drug” as used herein relates to a therapeutically active agent, also commonly called active pharmaceutical ingredient (API), as well as to a combination of plural such therapeutically active substances. The term also encompasses diagnostic or imaging agents, like for example contrast agents (e.g., MRI contrast agents), tracers (e.g., PET tracers) and hormones, that need to be administered in liquid form to the patient.

The term “drug substance” as used herein relates to a drug as defined above formulated or reconstituted in a form that is suitable for administration to the patient. For example, besides the drug, a drug substance may additionally comprise an excipient and/or other auxiliary ingredients. A particularly preferred drug substance in the context of the invention is a drug solution, in particular a solution for oral administration, injection or infusion.

The term “drug product” as used herein relates to a finished end product comprising a drug substance or a plurality of drug substances. In particular, a drug product may be a ready to use product having the drug substance in an appropriate dosage and/or in an appropriate form for administration. For example, a drug product may include an administration device such as a prefilled syringe or the like.

The medical delivery device further comprises a rotation inhibiting formation coupled to at least one of the barrel assembly and the rod assembly. In this connection the term “coupled” relates to a direct or indirect coupling or connection. The rotation inhibiting formation defines an extra rotation resistance affecting rotation of the barrel assembly and the rod assembly relative to each other.

By providing an extra rotation resistance it can be achieved that a reverse movement of the barrel assembly and the rod assembly relative to each other after stopping dosing rotation is prevented. More specifically, it can be prevented that a negative pressure inside the dosage chamber pulls back the rod assembly. Thus, an improved accurate dosing can be achieved. Further, accuracy of indicating the dosage amount can be increased.

Thereby, the extra rotation is not intended to block or prevent manual rotation of the barrel assembly and the rod assembly relative to each other but to increase the resistance of rotation still allowing manual rotation.

Thus, preferably, the rotation inhibiting formation is configured to define the extra rotation resistance such that the barrel assembly and the rod assembly are manually rotatable relative to each other against the extra rotation resistance. Like this, without preventing manual dosing, the rotation inhibiting formation can prevent unintended rotation which may be caused negative pressure inside the dosage chamber.

In other words, the rotation inhibiting formation preferably is configured to provide the extra rotation resistance during rotation of the barrel assembly and the rod assembly relative to each other, i.e., during manual dosing. Thus, rather than preventing manual dosing, the rotation inhibiting formation increases resistance of rotation during dosing.

Preferably, the barrel assembly and the rod assembly define an intrinsic rotation resistance affecting rotation of the barrel assembly and the rod assembly relative to each other, and a total rotation resistance being the sum of the intrinsic rotation resistance and the extra rotation resistance has to be overcome to rotate the barrel assembly and the rod assembly relative to each other.

The term “intrinsic” in connection with the barrel assembly and the rod assembly relates to features belonging to the essential nature or constitution of the barrel assembly and the rod assembly as well as other components of the medical delivery device interacting with the barrel assembly and/or the rod assembly. In particular, the intrinsic rotation resistance may be constituted by all impacts acting against rotation of the barrel assembly and the rod assembly, wherein these impacts are provided by the given structure of the medical delivery device. Such impacts may include friction acting between the first and second thread arrangements, friction acting upon linear movement of the rod assembly in the medical delivery device and others.

The total rotation resistance is not dimensioned to block or prevent manual rotation of the barrel assembly and the rod assembly relative to each other but to provide sufficient resistance in order to prevent unintended rotation. Thus, preferably, the barrel assembly, the rod assembly and the rotation inhibiting formation are configured to define the total rotation resistance such that the barrel assembly and the rod assembly are manually rotatable relative to each other against the total rotation resistance.

Thereby, preferably the total rotation resistance defines a resistance force acting on the rod assembly, e.g. when linearly moving the plunger rod element in the hollow interior to decrease the volume of the dosage chamber, wherein, when linearly moving the plunger rod element of the rod assembly to increase the volume of the dosage chamber by rotating the rod assembly and the barrel assembly relative to each other, a negative pressure is generated in the dosage chamber which defines a pulling force acting on the rod assembly and potentially casing back rotation, and wherein the rotation inhibiting formation is configured such that the resistance force is higher than the pulling force. Like this, it can be achieved that by means of the rotation inhibiting formation the pulling force is compensated. In particular, the pulling force resulting from the intrinsic structure of the medical delivery device can be compensated by the rotation inhibiting formation. Thus, the negative effects of the pulling force on dosing accuracy can be reduced or even eliminated.

The rotation inhibiting formation preferably is configured such that a relative movement between the rod assembly and the barrel assembly is prevented when varying the volume of the dosage chamber is stopped. Like this, after dosing, any unintended rotation or movement can be prevented. This allows to assure than rotation of the rod assembly and the barrel assembly relative to each other exclusively happens upon manual rotation or operation and upon other effects such us an under pressure in the dosage chamber.

The rotation inhibiting formation may be embodied in a broad variety of variants. For example, it may comprise a clamping structure, or electric or magnetic means to increase rotation resistance. The different variants, embodiments and techniques to embody the rotation inhibiting formation described may be combined such that a sophisticated and accurate increase of the rotation resistance results.

In a preferred embodiment of the rotation inhibiting formation protrusions are provided which are configured to increase friction upon rotation of the barrel assembly and the rod assembly relative to each other. The protrusions can be embodied as teeth in a sharp or rounded shape, as prongs or in any other suitable form. In addition to increasing resistance such protrusion can also provide a tactile feedback representing the rotation of the barrel assembly and the rod assembly relative to each other. Like this, dosing, and more specifically the amount of rotation can be perceived by a user.

Thereby, the rotation inhibiting formation preferably comprises a bearing member equipped with the protrusions. Such bearing member may be an efficient implementation of the rotation inhibiting formation or one component thereof. In particular, the bearing member can efficiently be coupled in a torque proof manner to either the rod assembly or the barrel assembly. The bearing member preferably is sleeve shaped and the protrusions form an axial end of the sleeve. Like this, the bearing member can efficiently be coupled to one of the rod and barrel assemblies and the protrusions can abut the other one of the rod and barrel assemblies. Like this, an increased friction between the rod assembly and the barrel assembly upon relative rotation can efficiently be achieved.

The protrusions preferably abut the barrel assembly, and the barrel assembly and the protrusions preferably are rotatable relative to each other. Like this, an efficient implementation can be achieved particularly in embodiments where the barrel assembly is rotated. Thereby, when the bearing member is provided, the barrel assembly is rotatable relative to the bearing member.

The rotation inhibiting formation preferably comprises counter-protrusions configured to interact with the protrusions upon rotation of the barrel assembly and the rod assembly relative to each other. Like this, resistance upon relative rotation and tactile recognition can be further increased. Thereby, the barrel assembly preferably is equipped with the counter-protrusions.

Preferably, the barrel assembly comprises a dial member accessible from outside the medical delivery device to rotate the barrel assembly relative to the rod assembly. The dial member can be integrally embodied with the hollow interior such that the hollow interior is part of the dial member. Advantageously, it is a single component or part. Such dial member allows a convenient dosing operation.

Preferably, the barrel assembly comprises a chamber body element having the hollow interior. The chamber body may have a cylinder portion in which the hollow interior is arranged. It can be integrally embodied with the dial member. However, preferably the dial member and the chamber body are connected or coupled in a torque proof manner, when the medical deliver device is arranged to rotate the barrel assembly and the rod assembly relative to each other.

Preferably, a first one of the first thread arrangement of the barrel assembly and the second thread arrangement of the rod assembly has a thread and a second one of the first thread arrangement of the barrel assembly and the second thread arrangement of the rod assembly has an engaging element configured to engage the thread. The engaging element can be any element suiting the thread for engagement. For example, the engaging element can be a pin or a similar protrusion extending into the thread. Advantageously, at least two engaging elements are provided for achieving a uniform and robust transformation of the rotational movement into a straight-line movement of the plunger rod element. The thread can be equipped with a friction increasing structure of the rotation inhibiting formation such as a material having a comparably high friction. For example, the thread can be provided with a rubber foil as friction increasing structure.

However, in a preferred embodiment of the rotation inhibiting formation an engagement friction increasing member is provided and the engaging element is equipped with the engagement friction increasing member. The engagement friction increasing member can be a geometric shape generating an increased friction between the engaging element and the thread, when the two travel along each other. For example, such shape can be a substantial roughness or teeth. Alternatively or additionally, the friction increasing member can be a piece of a material having a substantial higher friction than the material the engaging element is made of. For example, the engagement friction increasing member can be a piece of rubber or similar material connected to the engaging element.

Preferably, the medical delivery device comprises a housing part housing the barrel assembly and the rod assembly, wherein the rotation inhibiting formation has a housing friction increasing member arranged to increase friction between the housing part and at least one of the barrel assembly and the rod assembly. Similarly as the engagement friction increasing member also the housing friction increasing member can be a geometric shape such as a substantial roughness or teeth, or a piece of a material having a substantial higher friction than the material the housing part is made of such as a piece of rubber or similar material.

Thereby, the housing part, the rod assembly or the barrel assembly preferably is equipped with the housing friction increasing member. This allows for an efficient implementation.

Preferably, the plunger rod element of the rod assembly comprises a stopper member arranged in the hollow interior of the barrel assembly. Such stopper member is provided for sealing the dosage chamber. It typically is made of a comparably soft elastic material such as rubber and is dimensioned to be slightly compressed when being arranged in the hollow interior. Like this, a seal dosage chamber can be provided. The friction between the stopper member and the barrel assembly usually is comparably high.

Preferably, the medical device comprises a delivery needle and a vial spike configured to pierce a cover of a vial, wherein in a dosing status a fluid passageway is established through the vial spike and the delivery needle into the hollow interior of the barrel assembly. Thereby, in the dosing status, the delivery needle preferably extends into the vial spike.

In this context, the term “delivery needle” relates to a needle intended and provided for delivery of a drug substance to a patient. Thus, the delivery needle is the needle intended for administration of the drug substance.

Such arrangement of the fluid passageway allows for an efficient dosing and a compact design of the medical delivery device.

### BRIEF DESCRIPTION OF THE DRAWINGS

The medical delivery device according to the invention is described in more detail hereinbelow by way of an exemplary embodiment and with reference to the attached drawings, in which:

FIG. 1 shows a perspective view of single disassembled components of an embodiment of the medical delivery device according to the invention;

FIG. 2 shows a perspective view of a chamber body of a barrel assembly of the medical delivery device of FIG. 1;

FIG. 3 shows a side view of the chamber body of FIG. 2;

FIG. 4 shows a cross sectional view of the chamber body of FIG. 2;

FIG. 5 shows a perspective view of a rod assembly of the medical delivery device of FIG. 1;

FIG. 6 shows a perspective view of the chamber body of FIG. 2 and the rod assembly of FIG. 5 while being put together;

FIG. 7 shows a perspective view of the chamber body of FIG. 2 and the rod assembly of FIG. 5 in an assembled state;

FIG. 8 shows a side view of a bearing member of the medical delivery device of FIG. 1;

FIG. 9 shows a cross sectional view of the bearing member along the line A-A of FIG. 6;

FIG. 10 shows a cross sectional perspective view of the medical delivery device of FIG. 1;

FIG. 11 shows a cross sectional side view of the medical delivery device of FIG. 1 before dosing; and

FIG. 12 shows a cross sectional side view of the medical delivery device of FIG. 1 after dosing and ready for injection.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

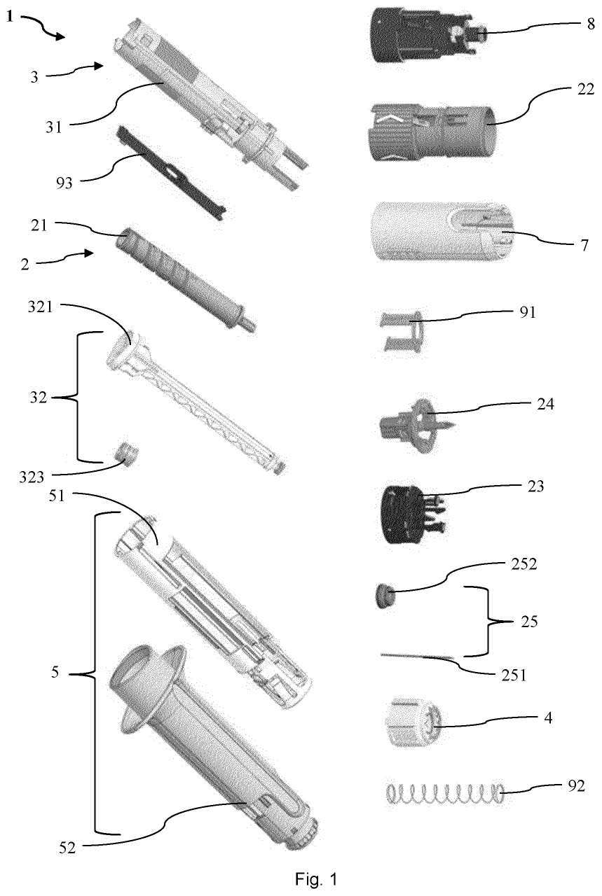

FIG. 1 shows the disassembled components of an embodiment of a medical delivery device 1 according to the invention. The medical delivery device 1 consists of a barrel assembly 2, a rod assembly 3, a bearing member 4, a housing part 5, a collar 7, a combining adapter 8, an activator ring 91, a spring 92 and a dose window member 93. The barrel assembly 2 has a chamber body 21, a dial member 22, a dial lock 23, a vial spike 24 and a needle arrangement 25 with a needle 251 as delivery needle and a needle seal 252. The rod assembly 3 comprises a plunger sleeve 31 and plunger rod element 32 with a stem member 321 defining a longitudinal axis and a rubber stopper 322.

The housing part 5 has a shuttle 51 and a safety sleeve 52 both being essentially cylindrical and having a hollow interior. The shuttle 51 is dimensioned to fit into the interior of the safety sleeve 52. The safety sleeve 52 is comparably rigid and does not include any moving portions at its outer surface. Furthermore, it is equipped with a finger flange near its back or proximal end. The shuttle 51 is equipped with structures and portions interacting with other components of the medical delivery device 1 when being assembled and used. Thus, the shuttle 51 can be referred to as the functional component of the housing part 5 as it provides functional interaction with other components.

The collar 7 is also essentially cylindrical and has a hollow interior. In an assembled state of the medical delivery device 1 it houses components of the medical delivery system 1 other than the components housed by the housing part 5. The dial member 22 provides an interface directly to a vial or to the combining adapted 8 which in turn may receive a vial.

The combining adapter 8 is embodied to receive a vial and to be releasably connectable to the other components of the medical delivery device 1. It is an optional component allowing for coupling or using a plurality of vials when dosing a liquid drug substance. Thus, the medical delivery device 1 can be used without the combining adapter 8 when the content of only one vial is to be dosed or with the combining adapter (or even a plurality thereof) when the contents of plural vials are to be dosed.

The activator ring 91 is embodied to activate the medical delivery device 1 when the combining adapter 8 is properly placed. In particular, the medical delivery device 1 is embodied such that no dosing can be performed when no vial or combining adapter 8 is received. Only after properly setting the vial or combining adapter 8 the medical delivery device is activated via the activator ring 91 such that dosing is possible.

The window member 93 is plank-like shaped and has an opening. It is provided to be shifted along the medical delivery device 1 during dosing such that the opening is adjacent to an appropriate marking. Like this, the appropriate marking is visible through the opening and a user can see the current dosing amount.

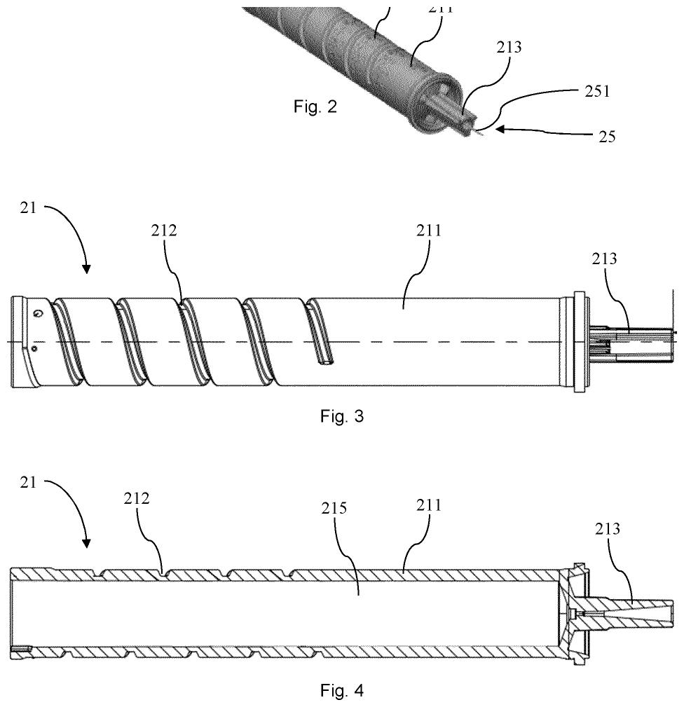

In FIG. 2 the chamber body 21 of the barrel assembly 2 is shown. It has a cylindrical portion 211 provided with a thread 212 as first thread arrangement. The outer surface of the cylindrical portion 211 is further equipped with markings or numbers indicating an amount dosed. More specifically, in an assembled state of the medical delivery device 1 the opening of the window member 93 moves along the markings or numbers of the cylindrical portion 211 when dosing such that the number or marking representing the current amount dosed into the medical delivery device 1 is visible.

The chamber body 21 further has a spout portion 213 extending from a front or distal end of the cylindrical portion 211. The spout portion 213 has an opening into which the needle 251 of the needle arrangement 25 is partially introduced. More specifically, the needle 251 is bonded to the spout portion 213 such that it extends out of the opening of the spout portion 213 into a distal direction.

As can be seen in FIG. 3 showing a side view of the chamber body 21, the thread 212 is embodied as groove helically extending about the cylindrical portion 211. Thereby, the thread 212 starts near a back or proximal end of the cylindrical portion 211 and extends as far as to about half of the length of the cylindrical portion 211.

FIG. 4 shows a cross section of the chamber body 21 of the barrel assembly 2. The chamber body 21 has a hollow interior 215 and is completely open at its left hand or proximal end. Thus, the hollow interior 215 is freely accessible from the left hand or proximal end of the chamber body 21. At an opposite right hand or distal end, the hollow interior 215 is closed by the spout portion 213. Thus, at the distal end of the cylindrical portion 211 the hollow interior 215 is accessible through or via the spout portion 213.

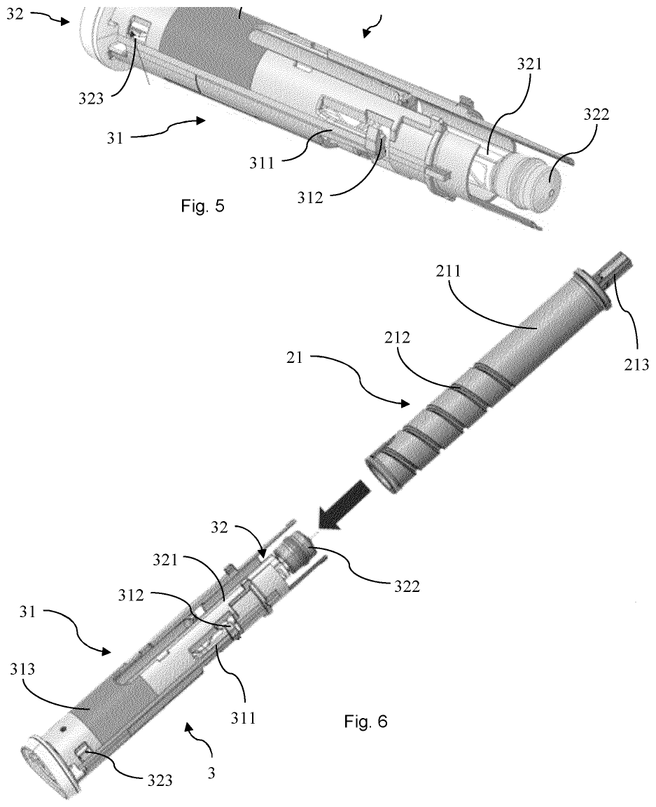

In FIG. 5 the rod assembly 3 is shown in an assembled state. In particular, the plunger sleeve 31 receives the plunger rod element 32 such that its stem member 321 extends through the plunger sleeve 31. Thereby, the rubber stopper 322 extends out of the plunger sleeve 31. At a back or proximal end, the stem member 321 passes over in a thumb receiving portion which abuts a back or proximal end of the plunger sleeve 31. Moreover, between the stem member 321 and the thumb receiving portion the rod element 32 has radially or outwardly extending protrusions extending through corresponding openings in a cylindrical section 313 of the plunger sleeve 31. Like this, the plunger sleeve 31 and the rod element 32 are locked to each other.

The plunger sleeve 31 further has two opposite resilient arms 311. At one axial end the arms 311 are passing over into the cylindrical section 313. At an opposite axial end, a pin 312 is arranged at each of the arms 311. The pins 312 axially extend towards the stem member 321 of the plunger rod element 32. Thereby, the pins 312 form a second thread arrangement.

FIG. 6 shows how the chamber body 21 is mounted to the rod assembly 3. The chamber body 21 is axially moved onto the rod element 32 such that the rubber stopper 322 is introduced though the open back or proximal end of the chamber body 21 into the hollow interior 215. When the chamber body 21 reaches the pins 312 of the plunger sleeve 31, the elastic arms 311 allow the pins 312 to be outwardly pressed such that the pins 312 can snap into the thread 212 of the chamber body 21.

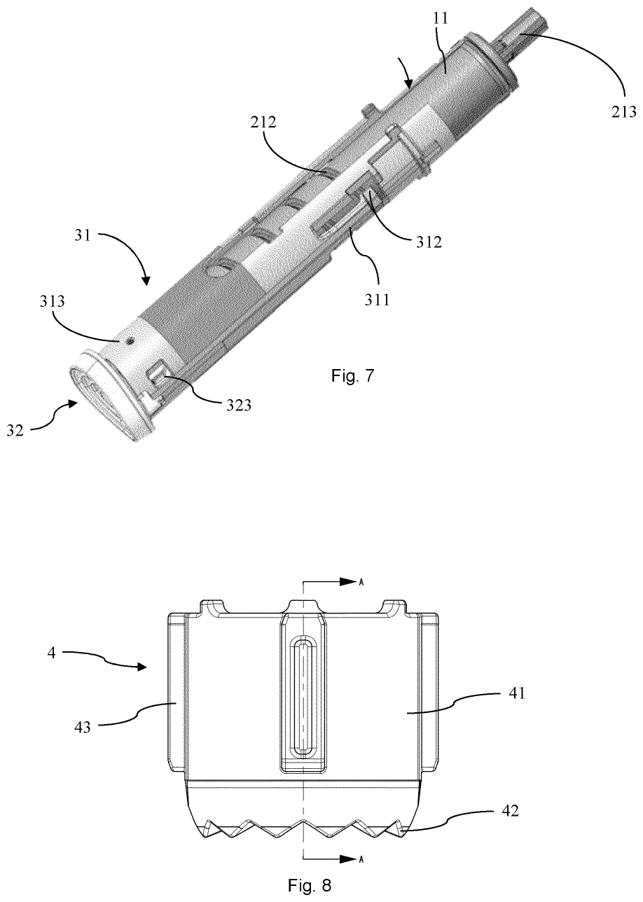

In FIG. 7 the rod assembly 3 and the chamber body 21 are shown when being finally assembled. Thereby, it can be seen that the pins 312 and the thread 212 engage such that rotation of the chamber body 21 and the rod assembly 3 relative to each other axially moves the rod assembly 3 relative to the chamber body 21.

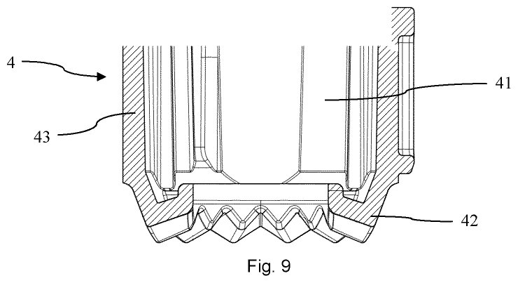

FIG. 8 shows a side view of the bearing member 4. It has a sleeve like body portion 41 from which four axial bars 42 laterally or radially extend. More specifically, at each 90° of the circumference of the body portion 41 one of the bars 42 extends. At its bottom end, the body 41 passes over into a protrusion portion which downwardly ends in a plurality of teeth 42 as protrusions. As can be seen in the cross-sectional view of FIG. 9, the body portion 41 is hollow and completely open in an upward direction. Also, the protrusion portion has a central opening such that the complete bearing member 4 has an axial passage.

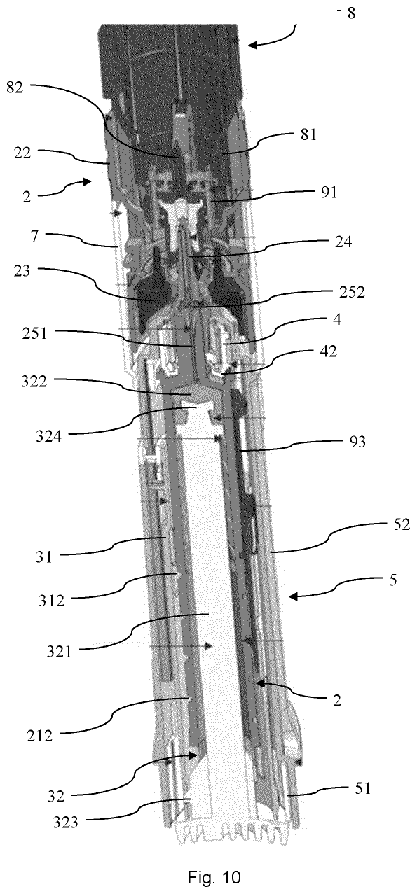

In FIG. 10, the completely assembled medical delivery device 1 is shown. Thereby, it can be seen that an upper or distal end of the stem member 321 of the plunger rod element 32 comprises a male form fit structure engaging a corresponding female form fit structure of the rubber stopper 322. Like this, the rubber stopper 322 is fixedly mounted to the stem member.

The combination of the chamber body 21 and the rod assembly 3 is received in the shuttle 51 of the housing part 5. The bearing member 4 is arranged inside the housing part 5 such that it is located between an axial end of the safety sleeve 52 and the chamber body 21. Thereby, the bars 42 are positioned in corresponding grooves of the safety sleeve 52 such that the bearing member 4 is torque proof relative to the housing part 5. The teeth 42 of the bearing member 4 abut the upper or distal end side of the cylindrical portion 411 of the chamber body 21.

The vial spike 24 is mounted on the spout portion 213 of the body chamber 21 in a torque proof manner such that the needle 251 extends into the vial spike 24. Around the vial spike 24 the dial lock 23 is mounted which in turn is mounted to the dial member 22. More specifically, the dial lock 23 is coupled to the vial spike 24 in a torque proof manner and to the dial member 22 to be torque proof up to a predefined torque. If the applied torque exceeds the predefined torque the dial lock 23 is rotatable relative to the dial member 22. Like this, the dial lock 23 provides an overload protection.

The dial lock 23 forms a vial seat into which the vial spike 24 upwardly extends. In the vial seat of the dial lock 23 the combining adapter 8 is arranged. The combining adapter 8 has another vial seat 81 with another spike 82. The spike 82 of the combining adapter 8 is in fluid tight connection with the vial spike 24, which in turn is in fluid tight connection with the needle 251 via the needle seal 252. The activator ring 91 has two legs extending into the vial seat 81 of the combining adapter 8.

The vial spike 24, the dial lock 23 and the vial seat of the dial member 22 are arranged inside the collar 7. The vial seat 81 and the spike 82 of the combining adapter 8 together with the activator ring 91 are arranged in the dial member 22. The dial member 22 and the collar 7 together with the components arranged therein form a unit which can be pulled off the housing part 5 together with the components arranged therein.

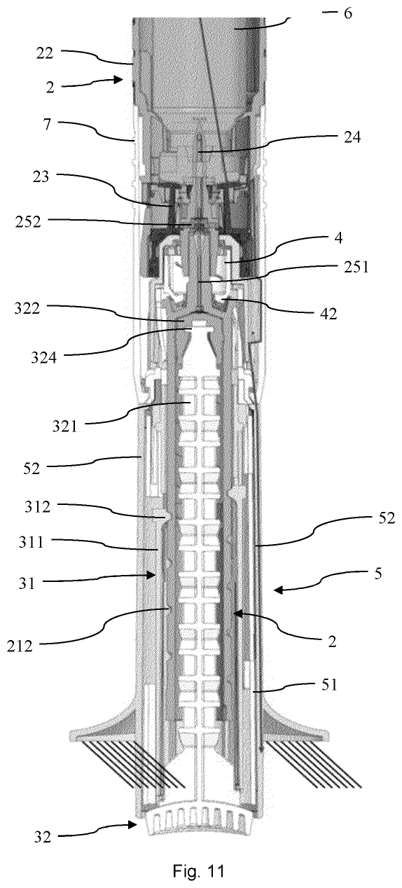

FIG. 11 shows the medical delivery device 1 in a dosing status. In particular, a vial 6 is pushed top down into the vial seat of the dial member 22 such that the vial spike 24 pierces a cover of the vial 6 and extends into an interior of the vial 6. Thereby, a fluid passageway is established from the interior of the vial 6 through the vial spike 24 and the needle 251 into the interior 215 of the chamber body 21. In the situation shown in FIG. 11 no combining adapter 8 is used.

For dosing, the dial member 22 is manually rotated by a user or operator about a longitudinal axis of the medical delivery device 1. Thereby, together with the dial member 22 the vial spike 24 is rotated which in turn rotates the chamber body 21 about the longitudinal axis. In contrast, the rod assembly 3 is not rotated. By such rotational movement of the chamber body 21 relative to the rod assembly 3, the pins 312 of the plunger sleeve 31 travel along the thread 212 of the chamber body 21 such that the stem member 321 is moved along the longitudinal axis relative to the chamber body 21.

The barrel assembly 2 and the rod assembly 3 define an intrinsic rotation resistance which affects relative rotation. In particular, the intrinsic rotation resistance comprises resistance caused by friction of the pins 312 travelling along the thread 212, by friction of barrel body contacting the plunger sleeve 31, by friction of the vial spike 24 contacting the dial lock 23, by friction of the dial member 22 contacting the collar 7, and by friction of the rubber stopper 322 contacting the chamber body 21. The bearing member 4 provides an extra rotation resistance particularly caused by the teeth 42 abutting the distal end of the cylinder portion 211 of the chamber body 21. For dosing, a total rotation resistance being the sum of the intrinsic rotation resistance and the extra rotation resistance has to be overcome. Thereby, the total rotation resistance defines a resistance force acting on the rod assembly 3, wherein when linearly moving the plunger rod element 32 of the rod assembly 3 to increase the volume of the dosage chamber 94 a negative pressure is generated in the dosage chamber 94 which defines a pulling force acting on the rod assembly 3, and wherein the rotation inhibiting formation 42 is configured such that the resistance force is higher than the pulling force. In particular, the bearing member 4 and its teeth 42 are configured such that a relative movement between the rod assembly 3 and the barrel assembly 2 is prevented when varying the volume of the dosage chamber 94 during or after dosing is stopped.

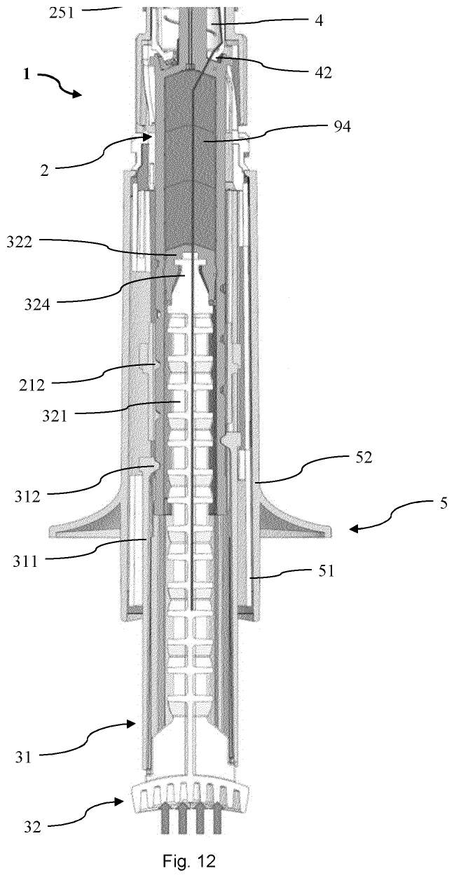

In FIG. 12 the medical delivery device 1 is shown after dosing in a delivery status. Thereby, by the stem member 321 being moved along the longitudinal axis relative to the chamber body 21 a dosage chamber 94 is generated in the hollow interior 215 of the chamber body 21. Moreover, the dosage chamber 94 is filled with a liquid drug substance withdrawn from the vial 6 during dosing. In particular, a volume of the dosage chamber 94 and, thus, an amount of liquid drug substance dosed is defined by the rotation described in connection with FIG. 11.

In the delivery status depicted in FIG. 12, the dial member 22 and the collar 7 together with the components arranged therein are pulled off the housing part 5 together with the components arranged therein. Thereby, the needle 251 is set free or exposed such that it can be pierced into a target. For delivery, the plunger rod element 32 is axially pushed such that the rubber stopper 322 forwards the liquid drug substance out of the medical delivery device 1 through the needle 251 by reducing the volume of the dosage chamber 94 to zero.

This description and the accompanying drawings that illustrate aspects and embodiments of the present invention should not be taken as limiting-the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1. A medical delivery device, comprising
a barrel assembly with a hollow interior and a first thread arrangement,
a rod assembly with a second thread arrangement and a plunger rod element having a longitudinal axis and extending into the hollow interior of the barrel assembly, and
a dosage chamber formed in the hollow interior of the barrel assembly with a variable volume limited by the plunger rod element of the rod assembly, and
a rotation inhibiting formation,
wherein the barrel assembly and the rod assembly are rotatable relative to each other, and
wherein the first thread arrangement of the barrel assembly and the second thread arrangement of the rod assembly engage such that rotation of the barrel assembly and the rod assembly relative to each other moves the rod assembly along the longitudinal axis of its plunger rod element causing the volume of the dosage chamber to vary,
wherein the rotation inhibiting formation is coupled to at least one of the barrel assembly and the rod assembly,
wherein the rotation inhibiting formation defines an extra rotation resistance affecting rotation of the barrel assembly and the rod assembly relative to each other.

2. The medical delivery device of claim 1, wherein the barrel assembly and the rod assembly define an intrinsic rotation resistance affecting rotation of the barrel assembly and the rod assembly relative to each other, and wherein a total rotation resistance being the sum of the intrinsic rotation resistance and the extra rotation resistance has to be overcome to rotate the barrel assembly and the rod assembly relative to each other.

3. The medical delivery device of claim 2, wherein the total rotation resistance defines a resistance force acting on the rod assembly, wherein, when linearly moving the plunger rod element of the rod assembly by rotating the barrel assembly and the rod assembly relative to each other to increase the volume of the dosage chamber, a negative pressure is generated in the dosage chamber which defines a pulling force acting on the rod assembly, and wherein the rotation inhibiting formation is configured such that the resistance force is higher than the pulling force.

4. The medical delivery device of claim 1, wherein the rotation inhibiting formation is configured such that a relative movement between the rod assembly and the barrel assembly is prevented when varying the volume of the dosage chamber is stopped.

5. The medical delivery device of claim 1, wherein the rotation inhibiting formation comprises protrusions configured to increase friction upon rotation of the barrel assembly and the rod assembly relative to each other.

6. The medical delivery device of claim 5, wherein the rotation inhibiting formation comprises a bearing member equipped with the protrusions and wherein the bearing member is sleeve-shaped and the protrusions form an axial end of the bearing member.

7. (canceled)

8. The medical delivery device of claim 5, wherein the protrusions abut the barrel assembly and wherein the barrel assembly and the protrusions are rotatable relative to each other.

9. The medical delivery device of claim 5, wherein the rotation inhibiting formation comprises counter-protrusions configured to interact with the protrusions upon rotation of the barrel assembly and the rod assembly relative to each other, wherein the barrel assembly is equipped with the counter-protrusions.

10. (canceled)

11. The medical delivery device of claim 1, wherein the barrel assembly comprises a dial member accessible from outside the medical delivery device to rotate the barrel assembly relative to the rod assembly.

12. The medical delivery device of claim 1, wherein the barrel assembly comprises a chamber body element having the hollow interior.

13. The medical delivery device of claim 11, wherein the barrel assembly comprises a chamber body element having the hollow interior and wherein the dial member and the chamber body element are connected in a torque proof manner, when the medical delivery device is arranged to rotate the barrel assembly and the rod assembly relative to each other.

14. The medical delivery device of claim 1, wherein a first one of the first thread arrangement of the barrel assembly and the second thread arrangement of the rod assembly has a thread and a second one of the first thread arrangement of the barrel assembly and the second thread arrangement of the rod assembly has an engaging element configured to engage the thread, and wherein the rotation inhibiting formation has an engagement friction increasing member and the engaging element is equipped with the engagement friction increasing member.

15. (canceled)

16. The medical delivery device of claim 1, comprising a housing part housing the barrel assembly and the rod assembly, wherein the rotation inhibiting formation has a housing friction increasing member arranged to increase friction between the housing part and at least one of the barrel assembly and the rod assembly.

17. The medical delivery device of claim 16, wherein the housing part, the rod assembly or the barrel assembly is equipped with the housing friction increasing member.

18. The medical delivery device of claim 1, wherein the plunger rod element of the rod assembly comprises a stopper member arranged in the hollow interior of the barrel assembly.

19. The medical delivery device of claim 1, wherein the rotation inhibiting formation is configured to define the extra rotation resistance such that the barrel assembly and the rod assembly are manually rotatable relative to each other against the extra rotation resistance.

20. The medical delivery device of claim 2, wherein the barrel assembly, the rod assembly and the rotation inhibiting formation are configured to define the total rotation resistance such that the barrel assembly and the rod assembly are manually rotatable relative to each other against the total rotation resistance.

21. The medical delivery device of claim 1, wherein the rotation inhibiting formation is configured to provide the extra rotation resistance during rotation of the barrel assembly and the rod assembly relative to each other.

22. The medical delivery device of claim 1, comprising a delivery needle and a vial spike configured to pierce a cover of a vial, wherein in a dosing status a fluid passageway is established through the vial spike and the delivery needle into the hollow interior of the barrel assembly.

23. The medical delivery device of claim 22, wherein in the dosing status the delivery needle extends into the vial spike.
